(19) United States
(12) Patent Application Publication
(10) Pub. No.: US 2002/0166179 A1
(43) Pub. Date: Nov. 14, 2002

(54) Process and composition for dying hair utilizing silicone fatty acid esters

(76) Inventors: Alan Wohlman, Northbrook, IL (US); Anthony J. O'Lenick JR., Dacula, GA (US)

Correspondence Address: A. J. O'Lenick, Jr., 2170 Luke Edwards Road, Dacula, GA, 30019, US

(21) Appl. No.: 09/777,022

(22) Filed: Feb. 6, 2001

(51) Int. Cl. A61K 7/13

(52) U.S. Cl. 8/405; 8/406; 8/408; 8/415; 8/421

(57) ABSTRACT

The present invention relates to a composition, process for preparation and use of a novel hair dying composition in the personal care industry. The composition when used in the dying process for hair results in a permanent coloration to the hair and improves manageability of the hair and minimizes damage from dying process. The use of silicone fatty esters having between 16 and 32 carbon atoms present in the ester portion of the molecule provide unexpected penetration of the hair by the dye, unexpected oxidative stability in the dye compositions, and unexpected color deposition to the hair.

### DESCRIPTION OF THE ART AND PRACTICES

The coloring of hair is one of the most important acts of adornment since the origin of man. The most effective coloring preparations on the market today are oxidative dyes. Almost all hair coloring is now performed with oxidation dye, both in the beauty salon and in homes. This type of dye dominates the market because the processes using these materials are quick, and lasting. These materials are called oxidative dyes because the dye must be placed on the hair, penetrate it and be oxidized, most commonly with hydrogen peroxide to make a color.

The dye when applied to the hair must be of low enough molecular weight to penetrate the hair, and be capable of being polymerized in the hair, in the presence of base and hydrogen peroxide, to form larger molecular weight colors. The chemical polymerization in the presence of base and peroxide is a coupling or condensation reaction. The base is an alkaline material selected from the group consisting of sodium hydroxide, potassium hydroxide, calcium hydroxide or the like. The base swells the hair and makes the penetration by the dye more rapid. It also participates in the condensation reaction.

The use of alkaline materials and hydrogen peroxide on the hair has a damaging effect upon the hair. Resulting in physical and chemical damage. However these additives have been necessary to (a) open up the hair t o make the penetration of the dye more effective and (b) to condense the dyes.

It is highly desirable to be able to deliver the dye to the hair and facilitate the penetration of the dye into the hair. We have surprisingly found that silicone functional ester derivatives having between 16 and 32 carbon atoms allows for the efficient and through penetration of the hair by the dyes, maintain color deposition and provide outstanding softness to treated hair. The preferred and optimum silicone ester is based upon meadowfoam seed oil. We have found surprisingly, that when the meadowfoam silicone ester is delivered in the hair dye improvements in color intensity, softness of hair and other improvements are made to the hair so treated when compared to hair dyed with the same formulation that does not contain the meadowfoam silicone ester. The meadowfoam silicone ester is added at concentrations of between 0.1% and 15% by weight with a preferred concentration between 1.0 and 5%.

### THE INVENTION

The present invention relates to a composition, process for preparation and use of a novel hair dying composition in the personal care industry. The composition when used in the dying process for hair results in a permanent coloration to the hair and improves manageability of the hair and minimizes damage from dying process.

The use of silicone fatty esters having between 16 and 32 carbon atoms present in the ester portion of the molecule provide unexpected penetration of the hair by the dye, unexpected oxidative stability in the dye compositions, and unexpected color deposition to the hair. In a preferred embodiment the ester is based upon meadowfoam seed oil. The improved functionality of silicone esters based upon this oil relates to the unique structure of the meadowfoam seed oil. The fatty distribution of the oil ranges from 20 to 22 carbons and has unsaturation in specific locations. The oil contains 97% by weight higher unsaturated alkyl groups. Typically, meadowfoam oil contains 60-65% of a twenty carbon mono-carboxy acid having one unsaturation between carbon 5 and 6. Additionally, it contains 12-20% of a twenty two carbon mono-carboxy acid having one unsaturation between either carbon 5 and 6, or carbon 13 and 14 and 15-28% of a twenty two carbon mono-carboxy acid having one unsaturation between both carbon 5 and 6, or carbon 13 and 14. The combination of the fact that there are 20 to 22 carbon atoms in the group leads to lack of volatility, the presence of unsaturation leads to liquidity and the fact that the di-unsaturated moieties are not conjugated leads to outstanding oxidative stability.

Meadowfoam oil is a triglyceride that conforms to the following structure:

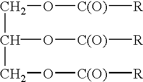

Wherein R is:

60-65% by weight —(CH₂)₃—CH═CH—(CH₂)₁₃—CH₃

12-20% by weight a mixture of

—(CH₂)₃—CH═CH—(CH₂)₁₅—CH₃ and

—(CH₂)₁₁—CH═CH—(CH₂)₇—CH₃ and

and 15-28% by weight

—(CH₂)₃—CH═CH—(CH₂)₆—CH—CH—(CH₂)₆—CH₃.

### OBJECT OF THE INVENTION

It is the objective of the present invention to provide a composition which contains (a) a silicone functional fatty acid ester (b) hair dye colors including main oxidation bases and coupling agents, (c) an oxidizing agent, and (d) a base selected from the group consisting of ammonia, NaOH and KOH. The composition is mixed together just prior to use and provides a very efficient dyeing process for hair. The ease of penetration, overall uniformity of color deposition and overall condition of the hair so treated is outstanding and here to fore unattainable using older technology.

### SUMMARY OF THE INVENTION

### DETAILED DESCRIPTION OF THE INVENTION

The compositions of the present invention are made up of the following components:

(a) a silicone functional fatty acid ester conforming to the following structure:

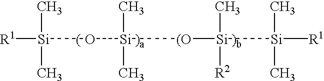

wherein:

a is an integer ranging from 1 to 200;

b is an integer ranging from 1 to 20;

R¹ and R₂ are selected from the group consisting of CH₃ and

—(CH₂)₃—O—(CH₂CH₂O)ₓ—(CH₂CH(CH₃)O)_{y}—(C₂CH₂O)_{z}—C(O)—R³

with the proviso that both R¹ and R₂ are not CH₃;

R³ is alkyl having 16 to 32 carbon atoms.

In a preferred embodiment R³ is derived from meadowfoam seed oil and has the following composition;

60-65% by weight —(CH₂)₃—CH═CH—(CH₂)₁₃—CH₃

12-20% by weight a mixture of

—(CH₂)₃—CH═CH—(CH₂)₁₅—CH₃ and

—(CH₂)₁₁—CH═CH—(CH₂)₇—CH₃ and

and 15-28% by weight

—(CH₂)₃—CH═CH—(CH₂)₆—CH═CH—(CH₂)₆—CH₃;

(b) hair dye colors including main oxidation bases and coupling agents selected from the group consisting of;

p-phenylenediamine,

p-aminophenoi hydrochloride,

2-amino-4-nitrophenol,

4-nitro-o-phenyienediamine,

o-aminophenol,

resorcinol,

pryogallol,

hydroquinone,

2-4-diaminophenol,

p-aminophenylene base,

2-nitro-o-phenylenediamine,

4,4-diaminoanisole sulfate,

4-nitro-o-phenyenediamine,

p-amiophenol,

p-toluylenediamine hydrochloride,

m-aminophenoi,

2,6-diaminopyridine,

6-chloro-4-nitro-2-aminophenol;

(c) an oxidizing agent, most commonly hydrogen peroxide

(d) a base selected from the group consisting of ammonia, NaOH, and KOH; and

(e) water.

The compositions of the present invention are used in a process for the dying of hair that comprises contacting the hair with an effective dyeing concentration of a composition that comprises:

(a) a silicone functional fatty acid ester conforming to the following structure:

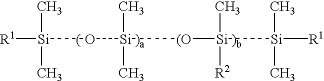

wherein:

a is an integer ranging from 1 to 200;

b is an integer ranging from 1 to 20;

R¹ and R₂ are selected from the group consisting of CH₃ and

—(CH₂)₃—O—(CH₂CH₂O)ₓ—(CH₂CH(CH₃)O)_{y}—(CH₂CH₂O)_{z}—C(O)—R³

with the proviso that both R¹ and R₂ are not CH₃;

R³ is alkyl having 16 to 32 carbon atoms.

In a preferred embodiment R³ is derived from meadowfoam seed oil and has the following composition;

60-65% by weight —(CH₂)₃—CH═CH—(CH₂)₁₃—CH₃

12-20% by weight a mixture of

—(CH₂)₃—CH═CH—(CH₂)₁₅—CH₃ and

—(CH₂)₁₁—CH═CH—(CH₂)₇—CH₃ and

and 15-28% by weight

—(CH₂)₃—CH═CH—(CH₂)₆—CH═CH—(CH₂)₆—CH₃;

(b) hair dye colors including main oxidation bases and coupling agents selected from the group consisting of;

p-phenylenediamine,

p-aminophenol hydrochloride,

2-amino-4-nitrophenol,

4-nitro-o-phenylenediamine,

o-aminophenol,

resorcinol,

pryogallol,

hydroquinone,

2-4-diaminophenol,

p-aminophenylene base,

2-nitro-o-phenylenediamine,

4,4-diaminoanisole sulfate,

4-nitro-o-phenyenediamine,

p-amiophenol,

p-toluylenediamine hydrochloride,

m-aminophenol,

2,6-diaminopyridine,

6-chloro-4-nitro-2-aminophenoi,

(c) an oxidizing agent, most commonly hydrogen peroxide

(d) a base selected from the group consisting of ammonia, NaOH, and KOH; and optionally and

(e) water.

In a preferred embodiment the compositions of the present invention are used in a process for the dying of hair that comprises contacting the hair with an effective dyeing concentration of a composition that comprises:

(a) between 0.1 and 5.0% by weight of a silicone functional fatty acid ester conforming to the following structure:

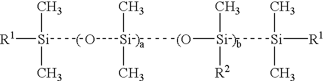

wherein:

a is an integer ranging from 1 to 200;

b is an integer ranging from 1 to 20;

R¹ and R₂ are selected from the group consisting of CH₃ and

—(CH₂)₃—O—(CH₂CH₂O)ₓ—(CH₂CH(CH₃)O)_{y}—CH₂CH₂O)_{z}—C(O)—R³

with the proviso that both R¹ and R₂ are not CH₃;

R³is alkyl having 16 to 32 carbon atoms.

In a preferred embodiment R³ is derived from meadowfoam seed oil and has the following composition;

60-65% by weight —(CH₂)₃—CH═CH—(CH₂)₁₃—(CH₃

12-20% by weight a mixture of

—(CH₂)₃—C═CH—(CH₂)₁₅—CH₃ and

—(CH₂)₁₁—CH═CH—(CH₂)₇—CH₃ and

and 15-28% by weight

—(CH₂)₃—CH═CH—(CH₂)₆—CH═CH—(CH₂)₆—CH₃;

(b) between 1.0% and 10.0% by weight hair dye colors including main oxidation bases and coupling agents selected from the group consisting of,

p-phenylenediamine,

p-aminophenoi hydrochloride,

2-amino-4-nitrophenol,

4-nitro-o-phenyienediamine,

o-aminophenol,

resorcinol,

pryogallol,

hydroquinone,

2-4-diaminophenol,

p-aminophenyiene base,

2-nitro-o-phenylenediamine,

4,4-diaminoanisole sulfate,

4-nitro-o-phenyenediamine,

p-amiopnenoi,

p-toluylenediamine hydrochloride,

m-aminophenoi,

2,6-diaminopyridine,

6-chloro-4-nitro-2-aminophenoi;

(c) between 1% and 15% by weight of an oxidizing agent, most commonly hydrogen peroxide;

(d) between 0.5% and 10.0% by weight of a base selected from the group consisting of ammonia, NaOH, and KOH; and

(e) between 97.5% and 65.0% by weight water.

### EXAMPLES

A. Silicone functional fatty acid ester conforming to the following structure:

The silicone ester useful in the preparation of compositions of the present invention are items of commerce and are available from Siltech Corporation Toronto Ontario Canada. They are the topic of U.S. Pat. No. 5,226,923 issued in 1993 incorporated herein by reference.

The silicone esters conforms to the following structure;

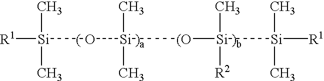

Class 1

R¹ is CH₃

R² is —(CH₂)₃—O—(CH₂CH₂O)ₓ—(CH₂CH(CH₃)O)_{y}—(CH₂CH₂O)_{z}—C(O)—R³

| Example | a | b | x | y | z |
|---|---|---|---|---|---|
| 1 | 0 | 1 | 0 | 10 | 10 |
| 2 | 2 | 4 | 0 | 0 | 10 |
| 3 | 20 | 6 | 0 | 0 | 0 |
| 4 | 50 | 10 | 20 | 10 | 10 |
| 5 | 100 | 15 | 20 | 0 | 20 |

Class 2

R¹ is —(CH₂)₃—O—(CH₂CH₂O)ₓ—(CH₂CH(CH₃)O)_{y}—(Ch₂CH₂O)_{z}—C(O)—R³

R² is CH₃

| Example | a | b | x | y | z |
|---|---|---|---|---|---|
| 6 | 200 | 20 | 0 | 0 | 20 |
| 7 | 200 | 1 | 20 | 20 | 20 |
| 8 | 100 | 4 | 5 | 5 | 13 |
| 9 | 50 | 6 | 0 | 0 | 0 |

(b) Hair dye colors including main oxidation bases and coupling agents selected from the group consisting of;

| Example | Compound |
|---|---|
| 10. | p-phenylenediamine, |
| 11. | p-aminophenol hydrochloride, |
| 12. | 2-amino-4-nitrophenol, |
| 13. | 4-nitro-o-phenylenediamine, |
| 14. | o-aminophenol, |
| 15. | resorcinol, |
| 16. | pryogallol, |
| 17. | hydroquinone, |
| 18. | 2-4-diaminophenol, |
| 19. | p-aminophenylene base, |
| 20. | 2-nitro-o-phenylenediamine, |
| 21. | 4,4-diaminoanisole sulfate, |
| 22. | 4-nitro-o-phenyenediamine, |
| 23. | p-amiophenol, |
| 24. | p-toluylenediamine hydrochloride, |
| 25. | m-aminophenol, |
| 26. | 2,6-diaminopyridine, |
| 27. | 6-chloro-4-nitro-2-aminophenol; |

(c) an oxidizing agent,

Example 28 H₂O₂ (35% in aqueous solution);

(d) a base selected from the group consisting of

Example 29 ammonia 28% in aqueous solution

Example 30 NaOH 50% in aqueous solution,

Example 31 KOH 45% in aqueous solution;

Formulations

Typical formulations used commercially contain a variety of ingredients. Addition of the compounds of the present invention to the formulated products just prior to application results in many desirable properties,

| Formulation #1 Red | |
|---|---|
| Component | % wt |
| WATER | 54.5 |
| OLEIC ACID | 4.0 |
| C12-15 PARETH-3 | 4.0 |
| AMMONIUM HYDROXIDE | 5.0 |
| BEHENTRIMONIUM CHLORIDE | 4.0 |
| C11-15 PARETH-9 | 4.0 |
| FRAGRANCE | 1.0 |
| STEARETH-21 | 3.0 |
| PROPYLENE GLYCOL | 1.0 |
| CETYL ALCOHOL | 3.0 |
| POLYQUATERNIUM-47 | 2.0 |
| PEG-150/STEARYL | 1.0 |
| COPOLYMER | |
| STEARYL ALCOHOL | 1.0 |
| ERYTHORBIC ACID | 0.5 |
| EDTA | 0.1 |
| SODIUM SULFITE | 0.1 |
| SODIUM METASILICATE | 0.1 |
| 4-AMINO-2- | |
| HYDROXYTOLULENE | 1.0 |
| P-AMINOPHENOL | 1.0 |
| MICA | 0.5 |
| IRON OXIDES | 0.1 |
| 1-NAPHTHOL | 1.0 |
| P-PHENYLENEDIAMINE | |
| TITANIUM DIOXIDE | 0.1 |
| 35% HYDROGEN PEROXIDE | 12.0 |
| Total | 100.0 |

Formulation of the Current Invention

| Formulation #2 Red | |
|---|---|
| Component | % wt |
| WATER | 53.5 |
| OLEIC ACID | 4.0 |
| C12-15 PARETH-3 | 4.0 |
| AMMONIUM HYDROXIDE | 5.0 |
| BEHENTRIMONIUM CHLORIDE | 4.0 |
| C11-15 PARETH-9 | 4.0 |
| FRAGRANCE | 1.0 |
| STEARETH-21 | 3.0 |
| PROPYLENE GLYCOL | 1.0 |
| CETYL ALCOHOL | 3.0 |
| POLYQUATERNIUM-47 | 2.0 |
| PEG-150/STEARYL | 1.0 |
| COPOLYMER | |
| STEARYL ALCOHOL | 1.0 |
| ERYTHORBIC ACID | 0.5 |
| EDTA | 0.1 |
| SODIUM SULFITE | 0.1 |
| SODIUM METASILICATE | 0.1 |
| 4-AMINO-2- | 1.0 |
| HYDROXYTOLULENE | |
| P-AMINOPHENOL | 1.0 |
| MICA | 0.5 |
| IRON OXIDES | 0.1 |
| 1-NAPHTHOL | 1.0 |
| P-PHENYLENEDIAMINE | |
| TITANIUM DIOXIDE | 0.1 |
| Meadowfoam Silicone Ester | 1.0 |
| (EXAMPLE 2) | |
| 35% HYDROGEN PEROXIDE | 12.0 |
| Total | 100.0 |

Formulation of the Current Invention

| Formulation #2 Red | |
|---|---|
| Component | % wt |
| WATER | 53.5 |
| OLEIC ACID | 4.0 |
| C12-15 PARETH-3 | 4.0 |
| AMMONIUM HYDROXIDE | 5.0 |
| BEHENTRIMONIUM CHLORIDE | 4.0 |
| C11-15 PARETH-9 | 4.0 |
| FRAGRANCE | 1.0 |
| STEARETH-21 | 3.0 |
| PROPYLENE GLYCOL | 1.0 |
| CETYL ALCOHOL | 3.0 |
| POLYQUATERNIUM-47 | 2.0 |
| PEG-150/STEARYL | 1.0 |
| COPOLYMER | |
| STEARYL ALCOHOL | 1.0 |
| ERYTHORBIC ACID | 0.5 |
| EDTA | 0.1 |
| SODIUM SULFITE | 0.1 |
| SODIUM METASILICATE | 0.1 |
| 4-AMINO-2- | 1.0 |
| HYDROXYTOLULENE | |
| P-AMINOPHENOL | 1.0 |
| MICA | 0.5 |
| IRON OXIDES | 0.1 |
| 1-NAPHTHOL | 1.0 |
| P-PHENYLENEDIAMINE | |
| TITANIUM DIOXIDE | 0.1 |
| Meadowfoam Silicone Ester | 1.0 |
| (EXAMPLE 6) | |
| 35% HYDROGEN PEROXIDE | 12.0 |

A scale of 1-5 was used to evaluate the various attributes of hair treated with a commercial formulation of hair dye and one using the same hair dye to which was added the silicone ester prior to use.(process of the present invention). I was defined as poor and 5 defined as excellent.

| | Standard | | |
|---|---|---|---|
| | Formulation | Present Invention | Present Invention |
| Attribute | Formulation 1 | Formulation 2 | Formulation 3 |
| Formulation | 5 | 5 | 4 |
| Stability | | | |
| Softness of hair | 3 | 5 | 5 |
| Color Intensity | 3 | 5 | 5 |

What is claimed:

1. A process for the simultaneous conditioning and dying of hair that comprises contacting the hair with an effective conditioning concentration of a composition that comprises:
(a) a silicone functional fatty acid ester conforming to the following structure: 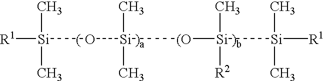
wherein:
a is an integer ranging from 1 to 200;
b is an integer ranging from 1 to 20;
R¹and R₂ are selected from the group consisting of CH₃ and
—(CH₂)₃—O—(CH₂CH₂O)ₓ—(CH₂CH(CH₃)O)_{y}—(CH₂CH₂O)_{z}—C(O)—R³
with the proviso that both R¹ and R₂ are not CH₃;
R³ is alkyl having 16 to 32 carbon atoms.
(b) hair dye colors including main oxidation bases and coupling agents selected from the group consisting of;
p-phenylenediamine,
p-aminophenoi hydrochloride,
2-amino-4-nitrophenol,
4-nitro-o-phenyienediamine,
o-aminophenol,
resorcinol,
pryogallol,
phydroquinone,
2-4-diaminophenol,
p-aminophenyiene base,
2-nitro-o-phenylenediamine,
4,4-diaminoanisole sulfate,
4-nitro-o-phenyenediamine,
p-amiophenol,
p-toluylenediamine hydrochloride,
m-aminophenol,
2,6-diaminopyridine,
6-chloro-4-nitro-2-aminophenol;
(c) an oxidizing agent, most commonly hydrogen peroxide
(d) a base selected from the group consisting of ammonia, NaOH, and KOH; and
(e) water.

2. A process of claim 1 wherein R³ is derived from meadowfoam seed oil and has the following composition;
60-65% by weight —(CH₂)₃—CH═CH—(CH₂)₁₃—CH₃
12-20% by weight a mixture of
—(CH₂)₃—CH═CH—(CH₂)₁₅—CH₃ and
—(CH₂)₁₁—CH═CH—(CH₂)₇—CH₃ and
and 15-28% by weight
—(CH₂)₃—CH═CH—(CH₂)₆—CH═CH—(CH₂)₆—CH₃.

3. A process for the simultaneous conditioning and dying of hair that comprises contacting the hair with an effective conditioning concentration of a composition that comprises:
(a) between 0.1 and 5.0% by weight of a silicone functional fatty acid ester conforming to the following structure: 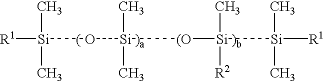
wherein:
a is an integer ranging from 1 to 200;
b is an integer ranging from 1 to 20;
R¹ and R₂ are selected from the group consisting of CH₃ and
—(CH₂)₃—O—(CH₂CH₂O)ₓ—(CH₂CH)CH₃)O)_{y}—(CH₂CH₂O)_{z}—C(O)—R³
with the proviso that both R¹ and R₂ are not CH₃;
R³ is alkyl having 16 to 32 carbon atoms.
(b) between 1.0% and 10.0% by weight hair dye colors including main oxidation bases and coupling agents selected from the group consisting of;
p-phenylenediamine,
p-aminophenol hydrochloride,
2-amino-4-nitrophenol,
4-nitro-o-phenylenediamine,
o-aminophenol,
resorcinol,
pryogallol,
hydroquinone,
2-4-diaminophenol,
p-aminophenylene base,
2-nitro-o-phenylenediamine,
4,4-diaminoanisole sulfate,
4-nitro-o-phenyenediamine,
p-amiophenol,
p-toluylenediamine hydrochloride,
m-aminophenol,
2,6-diaminopyridine,
6-chloro-4-nitro-2-aminophenol;
(c) between 1.0% and 15% by weight of an oxidizing agent, most commonly hydrogen peroxide;
(d) between 0.5% and 10% by weight of a base selected from the group consisting of ammonia, NaOH, and KOH; and
(e) between 97.5% and 65.0% by weight water.

4. A process of claim 3 wherein R³ is derived from meadowfoam seed oil and has the following composition;
60-65% by weight —(CH₂)₃—CH═CH—(CH₂)₁₃—CH₃
12-20% by weight a mixture of
—(CH₂)₃—CH═CH—(CH₂)₁₅—CH₃ and
—(CH₂)₁₁—CH═CH—(CH₂)₇—CH₃ and
and 5-28% by weight.

5. A process of claim 4 wherein said hair dye colors is o-aminophenol.

6. A process of claim 4 wherein said hair dye colors is resorcinol.

7. A process of claim 4 wherein said hair dye colors is pryogallol.

8. A process of claim 4 wherein said hair dye colors is hydroquinone.

9. A process of claim 4 wherein said hair dye colors is 4-nitro-o-phenylenediamine.

10. A process of claim 4 wherein said hair dye colors is 2-amino-4-nitrophenol.

11. A process of claim 4 wherein said hair dye colors is p-aminophenol hydrochloride.

12. A process of claim 4 wherein said hair dye colors is p-phenylenediamine.

13. A process of claim 4 wherein said hair dye colors is 24-diaminophenol.

14. A process of claim 4 wherein said hair dye colors is p-aminophenylene base.

15. A process of claim 4 wherein said hair dye colors is 2-nitro-o-phenylenediamine.

16. A process of claim 4 wherein said hair dye colors is 4,4-diaminoanisole sulfate.

17. A process of claim 4 wherein said hair dye colors is 4-nitro-o-phenyenediamine.

18. A process of claim 4 wherein said hair dye colors is p-amiophenol.

19. A process of claim 4 wherein said hair dye colors is 6-chloro-4-nitro-2-aminophenol.
